(19) United States
(12) Patent Application Publication
(10) Pub. No.: US 2004/0241097 A1
(43) Pub. Date: Dec. 2, 2004

(54) Diagnosis marker

(76) Inventor: Robert Hahn, Tullinge (SE)

Correspondence Address: BROWDY AND NEIMARK, P.L.L.C., 624 NINTH STREET, NW, SUITE 300, WASHINGTON, DC, 20001-5303, US

(21) Appl. No.: 10/478,958

(22) PCT Filed: Mar. 27, 2003
(86) PCT No.: PCT/SE03/00510

(30) Foreign Application Priority Data
Mar. 27, 2002 (SE) 0200932-2

(51) Int. Cl. A61K 49/00

(52) U.S. Cl. 424/9.52

(57) ABSTRACT

Use of a gas or gas precursor for the manufacture of a monitoring agent for a diganostic method for monitoring cardiac output in a mammal, including man, wherein said monitoring agent is a diagnostically acceptable gas in the gaseous state adapted for intravenous use, said gas being of such a nature, and being used in such an amount, that it is detectable via the expired breath from the mammal in question. An especially preferable gas is nitrous oxide. Monitoring agent for such a diagnostic method as well as such a diagnostic method.

### TECHNICAL FIELD

The present invention is within the field of diagnosing cardiac output in a mammal, including man. More specifically it relates to the use of a gas or gas precursor as a marker, which is detectable via the expired breath from said mammal.

### BACKGROUND OF THE INVENTION

Monitoring the cardiovascular system to determine myocardial performance is of primary importance in patient care. Such monitoring commonly begins with a determination of the heart rate and blood pressure of the patient. However, in the case of patients who are experiencing severe cardiac difficulties, additional diagnostic information about the operation of the heart is, often urgently, needed.

One important parameter in examining the condition of the heart is the cardiac output (CO), as said parameter is associated with the strength of the heart. More specifically, “cardiac output” is generally defined as the average of the total blood flow in the cirulatory system per unit time.

Today the monitoring of cardiac output is primarily performed by means of a thermodilution technique using a flow-directed catheter (Swan-Ganz catheter).

However, such a technique is invasive, the potential risk of e.g. hemorrhage, dysrhythmia or cardiac arrest being relatively high. Consequently, the use thereof is generally limited to specific clinical situations where the benefits far outweigh the risks. Furthermore, said technique is expensive and complicated and requires a highly trained technical personnel.

Therefore, there is a great demand for non-invasive techniques. One example of such a technique is a technique where ultrasound is utilized to monitor cardiac output. Such a technique is disclosed in e.g. U.S. Pat. No. 4,316,391. However, said technique is based on the creation of microbubbles to be ultrasonically imaged and is also very expensive and requires personnel with specific training.

Another technique is disclosed in Klocke F. J. et al. “Measurements of Cardiac Output Using Improved Chromatographic Analysis of Sulfur Hexafluoride (SF₆), Respiration Physiology, Vol 30, No 1-2, pp 99-107. Said technique is based on the use of a gas marker dissolved in a liquid, e.g. physiological saline, the detection being made by means of a gas chromatograph. Also this technique is complicated and requires trained personnel.

A specific example of still another non-invasive technique is the technique disclosed in WO 00/42908 (=EP 1 152 688). This document also contains an extensive survey of background art in this technical field, to which reference is also made concerning related art. However, the technique and apparatus disclosed in said document are rather complicated and are based on measurements of inspired and respired gases.

Reference is also made to WO 00/53087 (=EP 1 154 720), which discloses a method and an apparatus for determining the cardiac output of a patient. Although an indicator is injected into the blood, the method is based on the use of an indicator solution and at least one catheter, the determination being made on the basis of a change in a value of said indicator in the patient's bloodstream. Primarily, the method in question seems to be based on the thermal dilution technique. In other words, the principle is another than the principle behind the present invention and it is also rather complicated, as is generally the case for the prior art methods.

### DISCLOSURE OF THE INVENTION

The present invention is primarily based on the finding that when administering a gas to a mammal by intravenous injection said gas is detectable via the expired breath from said mammal and is directly, or indirectly, related to the cardiac output (CO) of the mammal in question. In other words, the gas is used as a marker for the transport of blood between the right ventricle and the lung. Thus, by measuring the concentration of gas in the expired breath from said mammal over time the cardiac output can be monitored via the pattern of the found concentration.

When for instance nitrous oxide is used as said gas in this way as a detectable marker, already small amounts thereof can be detected rapidly. In other words, the present invention enables the use of a new monitoring technique which is rapid as well as non-invasive and does not cause any discomfort to the patient.

In addition thereto, the new technique is very simple and cheap, as there will for instance not be needed any visual determinations by highly competent or trained personnel. No blood sampling or any other more or less complicated blood or flow measurements are needed.

In addition thereto, detection of e.g. exhaled nitrous oxide is very accurate and can be made with simple and even existing nitrous oxide monitors. For instance, such monitors are available in anaesthesia machines, which monitors may be used as such or easily converted into more accurate monitors. The monitoring can also be automatized.

The invention is of great importance in connection with care of very ill patents in cardiology (heart diseases) as well as in anaesthesia (surgery) and intensive care. Especially in a case where the patient is anaesthetized and ventilated mechanically the present invention enables a very simple technique adaptable to the existing major routine surgery.

Other objects of or advantages with the invention should be apparent to a person skilled in the art after having read the description below.

More specifically, according to a first aspect of the present invention, there is provided use of a gas or gas precursor for the manufacture of a monitoring agent for a diagnostic method for monitoring cardiac output in a mammal, including man, wherein said monitoring agent is a diagnostically acceptable gas in the gaseous state adapted for intravenous use, said gas being of such a nature, and being used in such an amount, that it is detectable via the expired breath from the mammal in question.

In other words, in addition to a gas which is decetable via the expired breath from a mammal, the invention is applicable also the use of a gas precursor, i.e. a substance that is the source of such a gas in connection with the diagnostic method referred to.

Both the gas and the gas precursor should be diagnostically acceptable.

The gas precursor is preferably a volatile liquid, i.e. liquid which is readily vaporizable at a relatively low temperature, e.g. below 70° C. or even below 40° C. or 30° C.

The gas is thus administered intravenously. Preferably injection is made itno the right atrium but it may also be possible to make the administration in a peripheral vein.

As was said above, the gas used as a monitoring agent in accordance with the present invention is detectable in the expired breath from the mammal in question. Accordingly, the gas should be used in such an amount that the concentration thereof in the expired breath from the mammal is detectable by the desired gas monitor. Typically this means the use of 0.1-10 μL of gas, preferably 1-5 mL thereof, especially when using N₂O as said gas. However, a proper amount is easily determined by a person skilled in the art such that the detection is adapted to the detecting device utilized. Generally this may mean that a detection level in the range of 1 to 2000 ppm is aimed at.

Although generally the characteristics of the gas or gas precursor can not be specified in exact figures, it should be easy for a person skilled in the art to screen candidates for gases or gas precursors without undue experimental work, as long as the gas or gas precursor is diagnostically acceptable and is of such a nature that it is detectable in the expired breath from the mammal in question. Guidance in this respect follows from the following examples of useful gases and gas precursors:

Nitrous oxide (N₂O):

noble gases, e.g. argon, krypton and xenon;

lower hydrocarbons, e.g. ethane, ethene and acetylene;

sulphur hexafluoride (SF₆); and

fluorinated lower hydrocarbons or hydrocarbon derivatives, e.g. volatile inhalation anesthetics such as sevoflurane (=fluoromethyl-2,2,2-trifluoro-1-(trifluoromethyl)ethyl ether.

Nitrous oxide is especially preferable.

According to a second aspect of the invention, or expressed in another way, there is also provided a monitoring agent for a diagnostic method for monitoring cardiac output in a mammal, including man, which is a diagnostically acceptable gas or gas precursor in the gaseous state for intravenous use, said gas being of such a nature, and being used in such an amount, that it is detectable via the expired breath from the mammal in question.

As to preferable embodiments of said monitoring agent reference is made to those preferable embodiments which have been presented in connection with the use described above. Thus, such preferable embodiments should be applicable also to the monitoring agent per se.

Still another aspect of the invention is represented by a method of monitoring cardiac output in a mammal, including man, which comprises administering intravenously to said mammal a monitoring agent as defined above and monitoring the expired breath from said mammal by means of a gas detector to detect the gas therein.

Also in this case all preferable embodiments are applicable which have been described above in connection with the use.

The gas monitoring device, or detector, to be used in connection with the present invention could be selected among previously known gas detectors used in other connections or easily modified therefrom.

As concerns the detection, however, a closed circuit should preferably be utilized to make sure that the gas is not directly expired when passing the lungs. The patient should inhale and exhale for a few minutes until a steady state is obtained where the gas is evenly distributed in the blood. An adsorbent, e.g. soda lime, can be used to adsorb the carbon dioxide and some oxygen gas can be added to counteract hypoxia.

In the case of nitrous oxide an equilibrium thereof is typically created within 2 minutes.

The level of detected gas in inversely proportional to cardiac output.

The theory is that when breathing occurs at a normal rate, mass balance calculations show that substantially all injected N₂O is eliminated by breathing. If ventilation is low or rebreathing is performed, the concentration of gas is increased until it equals the arterial blood. Further injected gas enters the pulmonary venous blood and reaches the systemic circulation. The operation should be stopped when a safe equilibrium is reached in order to avoid loading of gas in peripheral tissues. Some loading of gas does not matter much but the calculations should then be based on the difference between baseline and steady stet concentrations of gas.

The operation is generally started with a bolus injection to fill the lungs and the breathing circuit with gas, and thereby to reduce the time required until steady state is achieved. A precise dosing of the bolus is not required. On the contrary the gas administration following thereupon should be properly controlled.

### FIGURE

The accompanying FIG. 1 is a graph showing monitoring gas (N₂O) concentration versus time after intravenous injection of said gas;

FIG. 2 is a plot of cardiac output versus steady state concentration of N₂O for one guinea pig; and

FIG. 3 is a plot of cardiac output versus steady state concentration of N₂O for another guinea pig.

### EXAMPLE

The invention will now be further decribed in a non-limiting way by the following working example.

Pure nitrous oxide was injected intravenously to guinea pigs in a bolus of 0.1-1.5 mL and followed by administration of 1 mL of the same gasper minute. A closed circuit with a CO₂-adsorber in the form of soda lime and with a small addition of oxygen (0.3 L/min) to the system was utilized.

Thus, nitrous oxide is cleared from the body via expired air from the lungs while using a rebreathing system. By continuous measurements of nitrous oxide in the expired air a pattern for said clearance is obtained. A steady state is soon reached and said steady state describes the moment when the concentration in the rebreathing system is equal to those of the smallest gas exchanging parts, i.e. the alveoli. The concentration of nitrous oxide in the alveoli is at an equilibrium with the nitrous oxide concentration in the pulmonary artery.

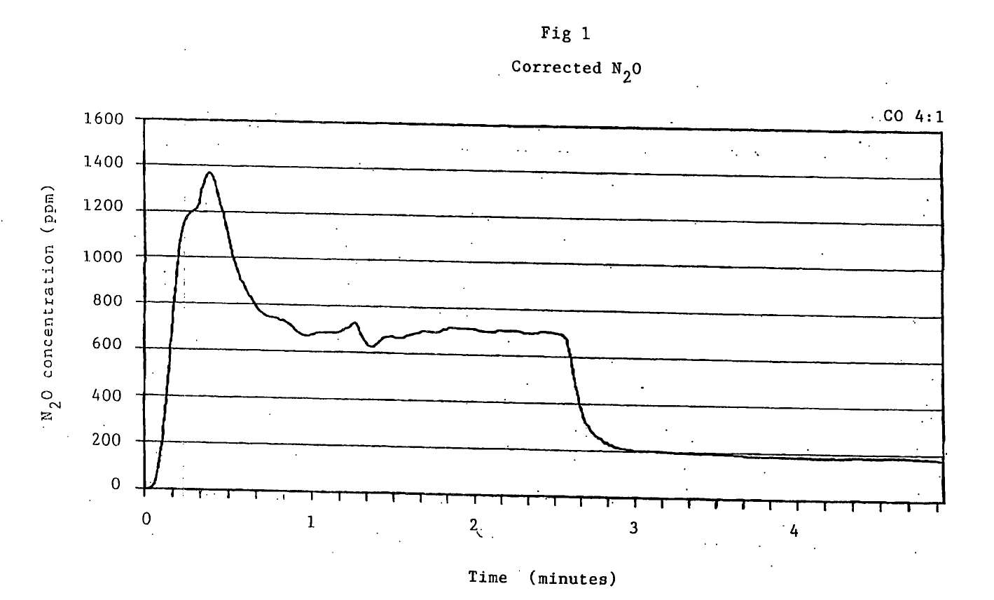

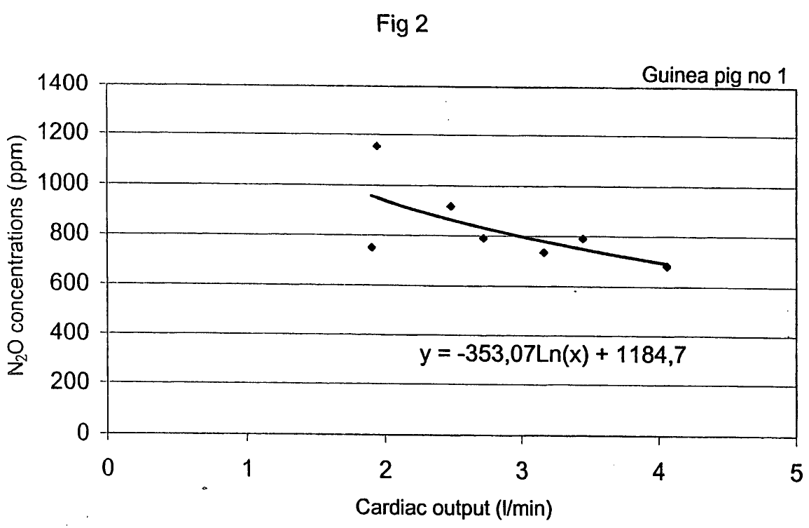

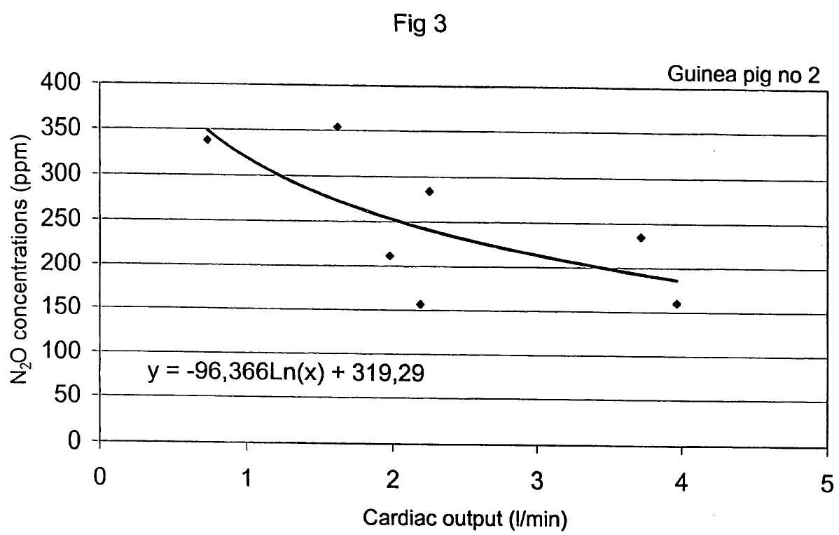

The result is initially shown in FIG. 1, from which it can be seen that an equilibrium of N₂O was achieved within two minutes. In FIG. 2 and FIG. 3. one can see the above-mentioned steady state concentration versus cardiac output. As has been described above, the level of N₂O is inversely proportional to cardiac output. That is, the lower the concentration of N₂O is in the pulonary artery, the higher is the cardiac output.

More specifically, FIGS. 1 and 2 show a plot between cardiac output, measured with the thermodilution method, and the concentrations of N₂O after steady state has been reached. Different administration rates were used for guinea pig 1 and 2 and thereby the different N₂O concentrations at steady state. Cardiac output was manipulated by making the guinea pig hyper and hypovolemic. The black line in the diagrams show a logarithmic regressions analysis of the plotted measurements.

As can be seen, there is a clear correlation between steady state concentrations of N₂O, and conventional measurement of cardiac output.

1-18. cancelled

19. A method of monitoring cardiac output in a mammal, including man, which comprises administering intravenously to said mammal, as a monitoring agent, a diagnostically acceptable gas in the gaseous state, said gas being of such a nature, and being used in such an amount, that it is detectable via the expired breath from the mammal in question and monitoring the expired breath from said mammal by means of a gas detector to detect the gas therein.

20. A method according to claim 19, which is used for monitoring cardiac output associated with cardiology, anaesthesia, or intensive care.

21. A method according to claim 20, wherein said gas is detected quantitatively.

22. A method according to claim 19, wherein said intravenous administration is injection into the right atrium.

23. A method according to claim 19, wherein said gas is nitrous oxide.

24. A method according to claim 19, wherein said gas is a noble gas.

25. A method according to claim 24, wherein said noble gas is selected from the group consisting of argon, krypton, and xenon.

26. A method according to claim 19, wherein said gas is a lower hydrocarbon.

27. A method according to claim 26, wherein said lower hydrocarbon is selected from the group consisting of ethane, ethene, and acetylene.

28. A method according to claim 19, wherein said gas is sulphur hexafluoride.

29. A method according to claim 19, wherein said gas precursor is a liquid.

30. A method according to claim 29, wherein said liquid is a fluorinated lower hydrocarbon or hydrocarbon derivative.

31. A method according to claim 30, wherein said fluorinated lower hydrocarbon or hydrocarbon derivative is sevoflurane.

32. A monitoring agent for a diagnostic method for monitoring cardiac output in a mammal, including man, which is a diagnostically acceptable gas or gas precursor in the gaseous state for intravenous use, said gas being of such a nature, and being used in such an amount, that it is detectable via the expired breath from the mammal in question.

33. A monitoring agent according to claim 32, wherein said gas is nitrous oxide.

34. A monitoring agent according to claim 32, wherein said gas is a noble gas.

35. A monitoring agent according to claim 34, wherein said noble gas is selected from the group consisting of argon, krypton, and xenon.

36. A monitoring agent according to claim 32, wherein said gas is a lower hydrocarbon.

37. A monitoring agent according to claim 36, wherein said lower hydrocarbon is selected from the group consisting of ethane, ethene, and acetylene.

38. A monitoring agent according to claim 32, wherein said gas is sulphur hexafluoride.

39. A monitoring agent according to claim 32, wherein said gas precursor is a liquid.

40. A monitoring agent according to claim 39, wherein said liquid is a fluorinated lower hydrocarbon or hydrocarbon derivative.

41. A monitoring agent according to claim 40, wherein said fluorinated lower hydrocarbon or hydrocarbon derivative is sevoflurane.

42. A method according to claim 19, wherein said gas is detected quantitatively.
